(19) United States
(12) Patent Application Publication
(10) Pub. No.: US 2024/0379195 A1
(43) Pub. Date: Nov. 14, 2024

(54) SYSTEMS AND METHODS FOR REDUCING SAMPLE SIZES

(71) Applicant: Regeneron Pharmaceuticals, Inc., Tarrytown, NY (US)

(72) Inventors: Rolando J. ACOSTA, Cambridge, MA (US); Emily R. Redington, Durham, NC (US); Erin E. Robertson, Chicago, IL (US); Jacek K. Urbanek, Eastchester, NY (US); Chenguang Wang, Potomac, MD (US); Henry Wei, Larchmont, NY (US); Matthew F. Wipperman, Brooklyn, NY (US)

(73) Assignee: Regeneron Pharmaceuticals, Inc., Tarrytown, NY (US)

(21) Appl. No.: 18/659,538

(22) Filed: May 9, 2024

Related U.S. Application Data
(60) Provisional application No. 63/501,111, filed on May 9, 2023.

(51) Int. Cl. G16H 10/20

(52) U.S. Cl. CPC G16H 10/20

(57) ABSTRACT

Aspects disclosed herein are directed to systems and methods including receiving external data including respective observed outcome data for a first set of subjects, training a machine learning framework based on a first subset of the external data to generate a trained machine learning framework, validating the trained machine learning framework using a second subset of the external data, extracting a baseline covariate based on validating the trained machine learning framework, determining a prognostic score for a first subject of a second set of subjects based on the baseline covariate, and classifying the first subject as a clinical trial subject based on the prognostic score. Further aspects include determining a correlation between a second observed outcome data of the second subset of the external data to a predicted outcome, and determining a reduced sample size for a study based on the correlation.

### CROSS-REFERENCED TO RELATED APPLICATIONS

This application claims the benefit of priority to U.S. Provisional Application No. 63/501,111, filed May 9, 2023, which is incorporated by reference herein in its entirety.

### TECHNICAL FIELD

Aspects of the present disclosure are directed to systems and methods for reducing sample sizes for clinical trials using machine learning. More specifically, aspects of the present disclosure are directed to systems and methods for adjusting for baseline covariates to reduce sample sizes for clinical trials to determine treatment effects.

### INTRODUCTION

Clinical trials and/or phenotyping for clinical trials is often limited by the population of individuals accessible for such clinical trials. Multiple factors such as availability of applicable individuals, technological resources, cost resources, and/or the like often limit the number of individuals accessible for such clinical trials. Accordingly, reducing the sample size (e.g., a number of individuals) and/or identifying individuals with applicable features (e.g., attributes) required for a clinical trial can increase efficiencies associated with the clinical trial and can also expand the number and types of clinical trials.

This introduction section is provided herein is for the purpose of generally presenting the context of the disclosure. Unless otherwise indicated herein, the materials described in this section are not prior art to the claims in this application and are not admitted to be prior art, or suggestions of the prior art, by inclusion in this section.

### SUMMARY OF THE DISCLOSURE

Aspects of the present disclosure relate to a method including receiving external data including respective observed outcome data for a first set of subjects, training a machine learning framework based on a first subset of the external data to generate a trained machine learning framework, validating the trained machine learning framework using a second subset of the external data, extracting a baseline covariate based on validating the trained machine learning framework, determining a prognostic score for a first subject of a second set of subjects based on the baseline covariate; and classifying the first subject as a clinical trial subject based on the prognostic score.

According to the method: the external data is received from one of a publicly available source, a previous clinical trial source, or a previously generated data source. The external data further includes feature data of a plurality of features. The method further includes harmonizing the external data. The machine learning framework includes one or more machine learning models. The machine learning framework is an ensemble framework. Validating the trained machine learning framework includes determining a correlation between a predicted second subset outcome and an observed second subset outcome. Validating the trained machine learning framework further includes comparing the correlation to a correlation threshold. Extracting the baseline covariate includes determining a most relied upon feature of a plurality of features. Extracting the baseline covariate includes determining a feature of a plurality of external data features that meets a weight threshold. Determining the prognostic score for the first subject includes providing a participant feature to one of a prognostic algorithm or a prognostic machine learning model.

Other aspects of the present disclosure relate to a system including a data storage device storing processor-readable instructions and a processor operatively connected to the data storage device and configured to execute the instructions to perform operations that include: receiving external data including respective observed outcome data for a first set of subjects, training a machine learning framework based on a first subset of the external data to generate a trained machine learning framework, validating the trained machine learning framework using a second subset of the external data, extracting a baseline covariate based on validating the trained machine learning framework, determining a prognostic score for a first subject of a second set of subjects based on the baseline covariate, and classifying the first subject as a clinical trial subject based on the prognostic score.

According to the system: The external data is received from one of a publicly available source, a previous clinical trial source, or a previously generated data source. The external data further includes feature data of a plurality of features. The machine learning framework comprises one or more machine learning models. Extracting the baseline covariate includes determining a most relied upon feature of a plurality of features. Extracting the baseline covariate includes determining a feature of a plurality of external data features that meets a weight threshold.

Other aspects of the present disclosure relate to a method including receiving external data including respective observed outcome data for a first set of subjects, training a machine learning framework based on a first subset of the external data to generate a trained machine learning framework, validating the trained machine learning framework using a second subset of the external data, determining a correlation between a second observed outcome data of the second subset of the external data to a predicted outcome data output by the trained machine learning framework based on the second subset of the external data, and determining a reduced sample size for a study based on the correlation. The correlation is based on a relationship between the second observed outcome data and the predicted outcome data. The reduced sample size is based on an original sample size of the study.

### BRIEF DESCRIPTION OF THE FIGURES

The accompanying drawings, which are incorporated in and constitute a part of this specification, illustrate various examples and, together with the description, serve to explain the principles of the disclosed examples and aspects.

Aspects of the disclosure may be implemented in connection with examples illustrated in the attached drawings. These drawings show different aspects of the present disclosure and, where appropriate, reference numerals illustrating like structures, components, materials, and/or elements in different figures are labeled similarly. It is understood that various combinations of the structures, components, and/or elements, other than those specifically shown, are contemplated and are within the scope of the present disclosure.

FIG. 1A is a plot of unadjusted outcome control and treatment data, in accordance with aspects of the present disclosure.

FIGS. 1B-1C are plots of adjusted outcome control and treatment data, in accordance with aspects of the present disclosure.

FIG. 1D is a table showing the results of the plots of FIGS. 1B-1C, in accordance with aspects of the present disclosure.

FIG. 2A is a flowchart for screening potential clinical trial participants, in accordance with aspects of the present disclosure.

FIG. 2B is a flowchart for determining a reduced sample size for a clinical trial, in accordance with aspects of the present disclosure.

FIG. 3 is a table of the top most predictive features of an outcome from a machine learning framework based on example external data, in accordance with aspects of the present disclosure.

FIG. 4 is a flow diagram depicting a general prognostic score generation procedure using an external data source, in accordance with aspects of the present disclosure.

FIG. 5A is a table including common primary and secondary outcomes based on hearing loss trials, in accordance with aspects of the present disclosure.

FIG. 5B is a table including common primary and secondary outcomes based on vestibular disease trials, in accordance with aspects of the present disclosure.

FIG. 5C is a flow diagram describing the hearing process, in accordance with aspects of the present disclosure.

FIG. 6 shows charts for hearing assessments, in accordance with aspects of the present disclosure.

FIG. 7A shows a diagram for characterizing hearing loss, in accordance with aspects of the present disclosure.

FIG. 7B shows a diagram depicting a cochlea, in accordance with aspects of the present disclosure.

FIG. 7C shows a diagram depicting a basilar membrane, in accordance with aspects of the present disclosure.

FIG. 7D depicts a correlation of a basilar membrane to a piano, in accordance with aspects of the present disclosure.

FIG. 7E shows a hearing audiogram, in accordance with aspects of the present disclosure.

FIG. 8 shows a table for enhancing auditory phenotyping for clinical trials, in accordance with aspects of the present disclosure.

FIG. 9A shows a table for external cohort data, in accordance with aspects of the present disclosure.

FIG. 9B shows a table for experimental external data, in accordance with aspects of the present disclosure.

FIG. 10A shows an example machine learning framework, in accordance with aspects of the present disclosure.

FIG. 10B shows how the example machine learning framework of FIG. 10A is used, in accordance with aspects of the present disclosure.

FIG. 11A shows a performance metrics plot for the example machine learning framework of FIG. 10A, in accordance with aspects of the present disclosure.

FIG. 11B shows an annotated performance metrics plot for the example machine learning framework of FIG. 10A, in accordance with aspects of the present disclosure.

FIG. 11C shows a plot depicting the accuracy of prediction of a hearing loss class for individuals, in accordance with aspects of the present disclosure.

FIG. 12 shows a flow diagram of an example auditory implementation, in accordance with aspects of the present disclosure.

FIG. 13 is a flow diagram for training a machine learning model, in accordance with aspects of the present disclosure.

FIG. 14 is an example computing environment, in accordance with aspects of the present disclosure.

Moreover, there are many aspects of the disclosed subject matter described and illustrated herein. The present disclosure is neither limited to any single aspect and/or aspects thereof, nor is it limited to any combinations and/or permutations of such aspects and/or implementations. Moreover, each of the aspects of the present disclosure, and/or aspects thereof, may be employed alone or in combination with one or more of the other aspects of the present disclosure and/or aspects thereof. For the sake of brevity, certain permutations and combinations are not discussed and/or illustrated separately herein. Notably, an aspect or implementation described herein as “exemplary” is not to be construed as preferred or advantageous, for example, over other aspects or implementations; rather, it is intended to reflect or indicate the aspects(s) is/are “example” aspects(s).

Notably, for simplicity and clarity of illustration, certain aspects of the figures depict the general structure and/or manner of construction of the various aspects disclosed herein. Descriptions and details of well-known features and techniques may be omitted to avoid unnecessarily obscuring other features. Elements in the figures are not necessarily drawn to scale; the dimensions of some features may be exaggerated relative to other elements to improve understanding of the example aspects. For example, one of ordinary skill in the art appreciates that the side views are not drawn to scale and should not be viewed as representing proportional relationships between different components. The side views are provided to help illustrate the various components of the depicted assembly, and to show their relative positioning to one another.

### DETAILED DESCRIPTION

Reference will now be made in detail to examples of the present disclosure, which are illustrated in the accompanying drawings. Wherever possible, the same reference numbers will be used throughout the drawings to refer to the same or like parts. The term “distal” refers to a portion farthest away from a user when introducing a device into a subject. By contrast, the term “proximal” refers to a portion closest to the user when placing the device into the subject. In the discussion that follows, relative terms such as “about,” “substantially,” “approximately,” etc. are used to indicate a possible variation of ±10% in a stated numeric value.

As used herein, the terms “comprises,” “comprising,” “includes,” “including,” or any other variation thereof, are intended to cover a non-exclusive inclusion, such that a process, method, article, or apparatus that comprises a list of elements does not include only those elements, but may include other elements not expressly listed or inherent to such process, method, article, or apparatus. The term “exemplary” is used in the sense of “example,” rather than “ideal.” In addition, the terms “first,” “second,” and the like, herein do not denote any order, quantity, or importance, but rather are used to distinguish an element or a structure from another. Moreover, the terms “a” and “an” herein do not denote a limitation of quantity, but rather denote the presence of one or more of the referenced items.

Aspects of the disclosed subject matter are directed to reducing the sample size required for conducting clinical trials and/or to identifying targeted features to screen individuals for clinical trials. As used herein, clinical trials may refer to studies that test one or more treatment effects or interventions such as for medical interventions, device interventions, surgical interventions, behavioral interventions, and/or the like. For example, such clinical trials may be implemented to determine if a treatment, intervention, and/or prevention, such as a technique, drug, diet, device, and/or process is safe and/or effective for a given population. Such clinical trials may include, but are not limited to, technology validation studies, genome wide association studies, expansion phenotyping studies, cohort studies, etc.

Aspects of the disclosed subject matter include adjusting for baseline covariates to reduce sample sizes required for a clinical trial and/or to identifying targeted features to screen individuals for clinical trials. As used herein, a baseline covariate may be a qualitative factor or quantitative variable (e.g., measured or observed) that is expected to influence a clinical outcome to be analyzed. Techniques disclosed herein may be implemented to reduce sample sizes for clinical trials and/or to optimize screening process for clinical trials. These techniques may be implemented such that reliable clinical trial outcomes can be obtained given the reduced sample sizes for clinical trials. Baseline covariates that correlate with a given clinical trial outcome (e.g., treatment/intervention outcomes) may be determined in accordance with the techniques disclosed herein. These baseline covariates may be used to target clinical trial populations, thereby reducing sample size in proportion with the respective correlation(s). The techniques disclosed herein provide benefits including reduction of resources (e.g., technical resources, medical resources, personnel, cost, etc.) to conduct clinical trials, increased efficiencies for clinical trials, allow increased use of digital endpoints to conduct clinical trials, and/or the like. Techniques disclosed herein include defining baseline covariates that correlate with a given clinical trial outcome. The defined baseline covariates may be used to improve the precision of, for example, treatment and/or intervention effect estimates, thereby reducing the required sample size of a corresponding clinical trial. For a given clinical trial, variation in an outcome associated with a treatment and/or intervention may be of interest whereas unintended variations may increase the complexity and/or noise associated with clinical trial results. Adjusting for baseline covariates that are correlated with an outcome removes or reduces unwanted variation in the outcome.

According to techniques disclosed herein, applicable digital composite covariates (e.g., pure tone averages (PTAs)) may be developed using external data sources. The external data sources may include publicly available data, previously generated data (e.g., based on previous clinical studies), casily collected data (e.g., based on limited resources), and/or the like. Such external data may be obtained without expending substantial cost or resources. For example, such external data may be obtained from publicly available studies such as government studies, non-profit studies, non-government organization studies and/or the like (e.g., Center for Disease Control and Prevention (CDC) data, World Health Organization (WHO) data, National Health and Nutrition Examination Survey (NHANES) data, National Health and Aging Trends Study (NHATS) data, United Kingdom (UK) Biobank data, Michael J. Fox Foundation data, Baltimore Longitudinal Study of Aging (BLSA) data, Atherosclerosis Risk in Communities (ARIC) study data, etc.). For example, NHANES is program of studies designed to assess the health and nutritional status of adults and children in the United States. It is operated by the US National Center for Health Statistics (NCHS), a branch of the Center for Disease Control and Prevention (CDC). It is unique in that it combines interviews and physical examinations of a sample of adults and children in the United States, and it occurs every two years since 1999. As another example, NHATS is a longitudinal study designed to assess the health, functioning, and well-being of older adults in the United States. It is led by investigators with support from the US National Institute of Aging (NIA), which part of the National Institutes of Health (NIH). It is unique in that it combines interviews, physical, and memory examinations of a sample of Medicare beneficiaries 65 and older, to assess the ways daily life changes as we age, and started in 2011.

Such external data may include, for example, biomedical data, biometric data, demographic data, physiological data, medical condition data, survey data, questionnaire data, and/or the like. Such external data may be retrieved from available databases and may not require additional or extensive data generation (e.g., in test or trial settings). Although auditory implementations are generally discussed herein, it will be understood that techniques disclosed herein may be applied to any implementation such as, but not limited to, any human or animal therapeutic categories, medical categories, treatment categories, etc. For example, techniques disclosed herein may be applied to analysis, studies, treatments, interventions, etc. associated with any applicable disease, medical condition, physiological condition, movement condition, organ condition, psychological condition, sense-based condition, electrical data, chemical data, and/or the like, or a combination thereof.

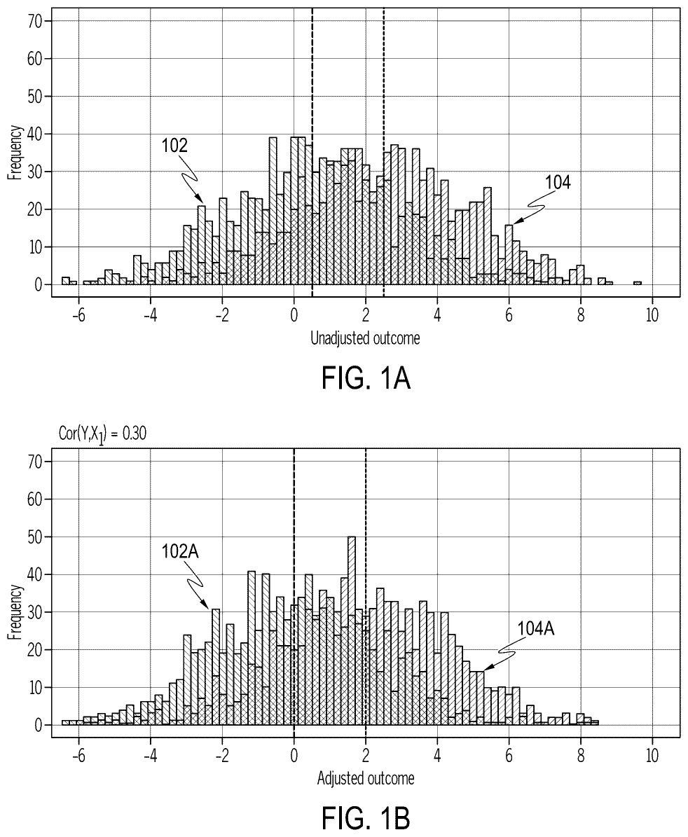

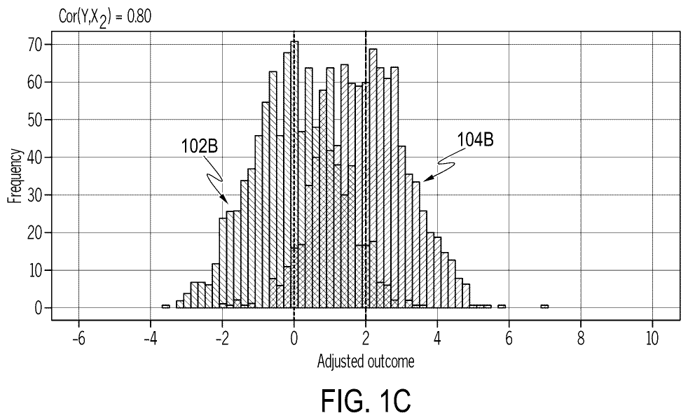

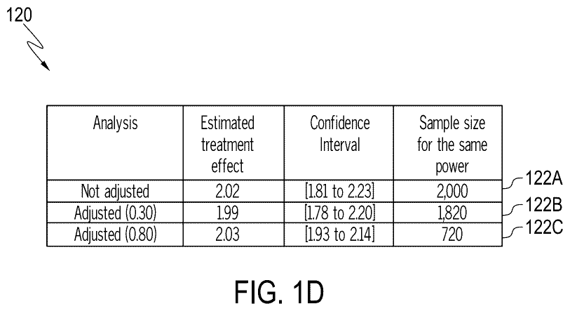

FIGS. 1A-1C include charts that exemplify adjusting for baseline covariates to reduce sample sizes required for a clinical trial and/or to screen individuals for a clinical trial. For a continuous outcome, an analysis that adjusts for a baseline covariate that has a correlation p with a given outcome may attain approximately the same results (e.g., same power) using a subset (e.g., approximately (1−ρ²) %) of the sample size needed in comparison to an unadjusted analysis. FIGS. 1A-1C show a simulation of this implementation based on 2,000 subjects with a 1:1 random split between control 102 and treatment 104 components. For the simulation of FIGS. 1A-1C, a true treatment effect is set to two. A first variable X1 having a correlation with the given outcome of 0.3 (FIG. 1B) and a second variable X2 having a correlation with the given outcome of 0.8 (FIG. 1C) is simulated. FIG. 1A shows an unadjusted outcome control 102 and treatment 104 data such that the outcome variation deviates from approximately −6 to approximately 10. FIG. 1B shows adjusted control 102A and treatment 104A data, adjusted for variable X₁ (having a correlation with the given outcome of 0.3). As shown in FIG. 1B, the outcome variability is reduced in comparison to FIG. 1A, such that the outcome variation deviates from approximately −6 to approximately 8. FIG. 1C shows adjusted control 102B and treatment 104B data, adjusted for variable X₂ (having a correlation with the given outcome of 0.8). As shown in FIG. 1C, the outcome variability is reduced in comparison to FIG. 1A and FIG. 1B, such that the outcome variation deviates from approximately −4 to approximately 6. The outcomes depicted in FIGS. 1B and 1C resulted in a reduction in sample size of 9% and 64% respectively.

FIG. 1D shows table 120 that includes the analysis associated with FIGS. 1A-1C. As shown in table 120, row 122A corresponds to the non-adjusted data of FIG. 1A. Row 122B corresponds to data adjusted for variable X₁ (having a correlation with the given outcome of 0.3). Row 122C corresponds to the data adjusted for variable X₂ (having a correlation with the given outcome of 0.8).

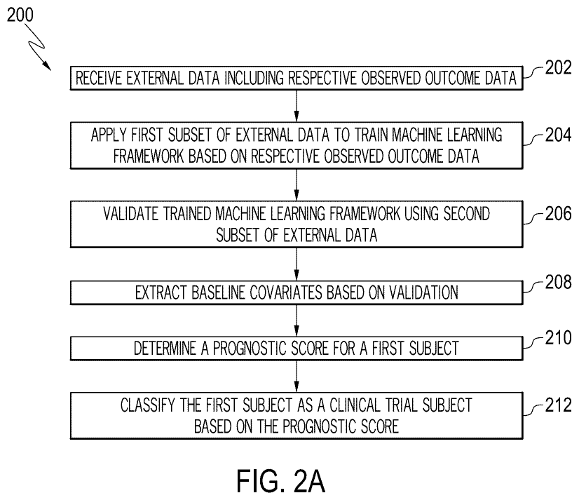

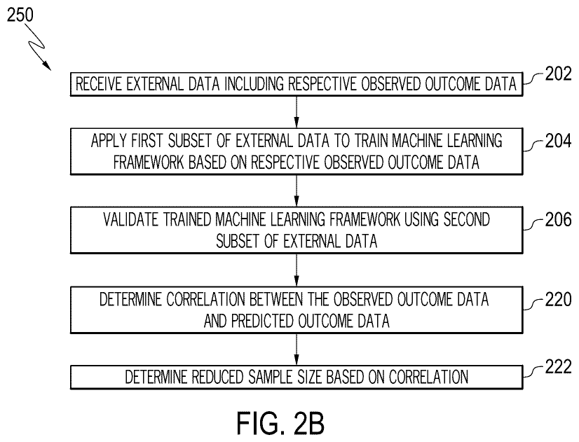

FIG. 2A shows a flowchart 200 for screening potential clinical trial participants based on a prognostic score. At step 202 of flowchart 200, external data including respective observed outcome data may be received. The external data may be received from a publicly available database, a previously generated data source, an easily accessible data source, etc., as discussed herein. The external data may include data for a plurality of individuals for respective plurality of features (e.g., covariates). Such features may include, for example, biomedical data, biometric data, demographic data, physiological data, medical condition data, survey results, questioner results, test results, assessments, and/or the like.

According to implementations of the present disclosure, the external data may be harmonized. The harmonization may include selecting applicable features, removing inapplicable features, wrangling and re-scaling data (e.g., to make covariate variables consistent across multiple cohorts or sets of individuals/data), and/or the like. The harmonization may allow use of the external data in accordance with techniques discussed herein. For example, as further discussed, the harmonization may result in the external data to be transformed into a format for training one or more machine learning models (e.g., of a machine learning framework).

The external data of step 202 may include feature data and observed outcome data. The observed outcome data may correspond to a given outcome associated with a clinical trial. For example, for auditory clinical trials, the observed outcome data may include hearing loss data (e.g., obtained based on a hearing quality test). Accordingly, the external data may include observed outcome data that may also be observed as an outcome of a planned clinical trial, in view of a treatment effect. Continuing the previous example, a clinical trial may observe hearing loss in view of an auditory treatment (e.g., a drug, a medical device, etc.). Accordingly, the external data including the observed hearing loss data (outcome data) may be received at step 202.

At step 204, a first subset of the external data received at step 202 may be applied to train a machine learning framework. As used herein, a “machine learning framework” may include one or more machine learning models. A machine learning framework implemented using one or more machine learning models may be implemented using an ensemble prediction framework that combines predictions from each of two or more machine learning models. The machine learning model(s) that contribute to the ensemble (e.g., ensemble members) may be the same type or different types and may or may not be trained on the same training data. Predictions from each machine learning model may be combined (e.g., an average, a median, a mode, another relationship, etc.) to obtain machine learning framework predictions. For aspects where two or more machine learning models are applied, outputs or features associated with one or more machine learning models may be weighted greater than outputs and/or features of one or more other machine learning models. “Machine learning models” are further discussed herein.

The first subset of external data may be data associated with a randomly selected or a specifically selected group of individuals (e.g., one or more cohorts). The first subset of external data may be used as training data for the machine learning framework. According to an implementation, the first subset of external data may be tagged data, where the tags correspond to the observed outcome data. Continuing the previous example, the first subset of external data may include features such as demographic data, biometric data, survey results, etc. associated with individuals (first subset of individuals) associated with the first subset of data. The first subset of data may also include hearing loss data (observed outcome data) for the first subset of individuals.

The machine learning framework may be trained using the first subset of external data in accordance with machine learning model training techniques further disclosed herein. As an example, supervised or semi-supervised training may be used to train one or more machine learning models of the machine learning framework based on the first subset of external data including the feature data and respective observed outcome data. A trained machine learning framework may be trained to output outcome data (e.g., test or production outcome data) based on input feature data (e.g., test or production feature data). The training at step 204 may result in the trained machine learning framework. According to an implementation, a third subset of the external data may be used to ensemble (e.g., stack) two or more machine learning models.

At step 206, a second subset of external data may be used to validate the trained machine learning framework. The second subset of external data may include, for example, data that is not used to train the machine learning framework at step 204. At step 206, the feature data (e.g., demographic data, biometric data, survey results, etc.) associated with individuals (second subset of individuals) associated with the second subset of data may be provided as inputs to the trained machine learning framework. The trained machine learning framework may output predicted outcomes for the second subset of individuals. Continuing the previous example, the feature data for the second subset of individuals may be provided to the trained machine learning framework. The trained machine learning framework may output indications regarding whether each of the second subset of individuals experience or are likely to experience hearing loss. An indication output by the machine learning framework may be a binary indication (e.g., true or false), a tier (e.g., level of hearing loss, hearing quality, etc.), a value (e.g., an amount of hearing loss), and/or the like.

The validation at step 206 may include comparing the predicted outcomes output by the trained machine learning model to the observed outcome data of the second subset of external data. Continuing the previous example, the hearing loss indications output by the trained machine learning model may be compared to the observed (known) outcomes (e.g., likely to experience hearing loss) for the second subset of individuals. A correlation (e.g., a correlation coefficient), as further discussed herein, may be determined for the trained machine learning framework's ability to accurately predict the outcome (e.g., hearing loss) for the second subset of individuals. The trained machine learning framework may be validated if the correlation exceeds a desired correlation threshold (e.g., approximately 0.5 for desired sample size reduction by 25%).

At step 208, if the machine learning framework is validated at step 206, then baseline covariates (e.g., features) of the external data (e.g., demographic data, biometric data, survey results, etc.) that most contribute to determining the predicted outcomes may be identified. For example, the machine learning framework (e.g., one or more machine learning models) may be analyzed (e.g., using an analysis software) to determine which features were most weighted when determining the predicted outcomes. According to an implementation, a number N of highest weighted features may be determined to be baseline covariates. According to another implementation, features weighted above a weight threshold may be determined to be baseline covariates.

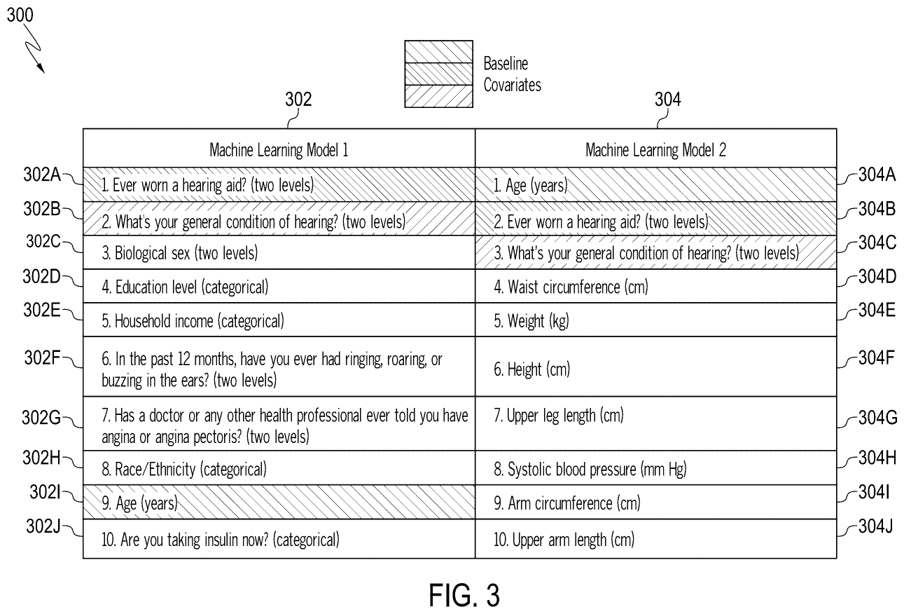

For example, FIG. 3 shows a table 300 of the top ten most predictive features of an outcome in the example external data used to train a machine learning framework (e.g., at step 204). The machine learning framework may include a Machine Learning Model 1 302 and a Machine Learning Model 2 304. As shown in FIG. 3, Machine Learning Model 1 302 may apply features 302A-302J during a training phase. Machine Learning Model 2 304 may apply features 304A-304J during a training phase. As also shown in FIG. 3, Machine Learning Model 1 302 may be analyzed to determine that feature 1 302A (Ever wore hearing aid?), feature 2 302B (General condition of hearing), and feature 9 3021 (age) contribute substantially (e.g., contribute more than the other features of Machine Learning Model 1) to predicting a hearing loss outcome. Machine Learning Model 2 may be analyzed to determine that features 1 304A (Age), feature 2 304B (Ever wore hearing aid?), and feature 3 304C (General condition of hearing) contribute substantially (e.g., contribute more than the other features of Machine Learning Model 2) to predicting a hearing loss outcome.

Accordingly, at step 208, features that most contribute to a validated machine learning framework accurately predicting a given outcome (above a correlation threshold) may be identified as baseline covariates. According to an example, such baseline covariates may correspond to features that most contributed (e.g., above a threshold that may be a numerical threshold or may be relative to other features) to modifying a weight, layer, bias, or synapse of a respective machine learning model during a training phase. As another example, such baseline covariates may correspond to features that most contributed (e.g., above a threshold that may be a numerical threshold or may be relative to other features) to predicting a clinical outcome. As discussed herein, these baseline covariates may most contribute to a given outcome (e.g., hearing loss) associated with a clinical trial for a treatment effect for predicting the given outcome. These baseline covariates may be used to screen potential clinical trial participants such that the corresponding clinical trial may be implemented in a more efficient manner. Screening potential clinical trial participants based on such identified baseline covariates may reduce the variability of the results of the clinical trial without biasing the same. Accordingly, screening for participants based on such baseline covariates may result in a more efficient clinical trial (e.g., by screening out participants based on such covariates), thereby reducing the sample size required for the clinical trial. Continuing the example discussed herein, identified baseline covariates may be used to screen (e.g., exclude) potential participants that are unlikely to experience hearing loss. The contemplated clinical trial outcome, according to this example, may be an effect on the degree of hearing loss based on a treatment effect (e.g., a drug, a medical device, etc.). Accordingly, excluding participants that are unlikely to experience hearing loss may provide for a more relevant/efficient clinical trial.

At step 210, according to an implementation, a prognostic score may be determined for potential clinical trial participants based on the baseline covariates extracted at step 208. The prognostic score may be determined for a potential clinical trial participant based on the presence, absence, and/or a value associated with one or more baseline covariates. For example, features associated with a potential clinical trial participant may be input into an algorithm or a prognostic score machine learning model. The prognostic score machine learning model may be the same as, part of, or separate from the machine learning framework discussed herein. At step 212, the algorithm or prognostic score machine learning model may output a prognostic score (e.g., a binary value, a tier, a numerical value, etc.) which may be compared to a threshold to screen potential clinical trial participants. The prognostic score may be used to determine if the potential clinical trial participant should be included in a given clinical trial or be excluded from the clinical trial.

FIG. 2B shows a flowchart 250 for determining a reduced sample size for a clinical trial. Steps 202-206 of FIG. 2B correspond to steps 202-206 of FIG. 2A. At step 220, a correlation (e.g., correlation coefficient) between predicted and observed outcome data of the external data received at step 202 may be determined, in accordance with the technique disclosed herein. The correlation may indicate the accuracy of the predicted outcomes output by the trained machine learning framework in comparison to the observed (known) outcome data of the second subset of external data.

At step 222, a reduced sample size for a clinical trial may be determined based on the correlation. The reduced sample size may be a relationship to a predicted or required sample size for the clinical trial. For example, the reduced sample size may be a percent or ratio of the predicted or required sample size for the clinical trial. As an example, the reduced sample size may be approximately (1−ρ²) % of the required sample size in an unadjusted analysis, where p represents the correlation (e.g., correlation coefficient) of the validated trained machine learning framework of step 206. According to an implementation, a number of clinical trial participants that meet the baseline covariates criteria (e.g., as determined at step 208 of FIG. 2A) may be based on the reduced sample size determined at step 222.

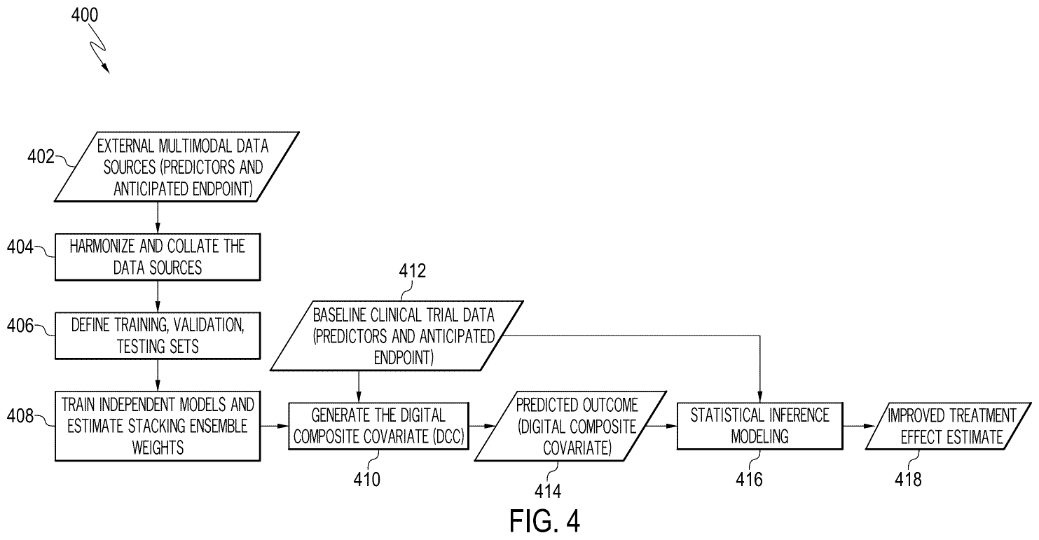

FIG. 4 shows a flow diagram 400 for an example implementation of the techniques disclosed herein. As shown in flow diagram 400, external data may be received from external multimodal data sources (e.g., having predictors (features) and anticipated endpoints (outcomes)) at 402. The external data may be harmonized and/or collated at 404. Training, validation, and/or test sets (e.g., first/second subsets of data) may be defined at 406. Independent models (e.g., a machine learning framework) and/or estimated stacking ensemble weights may be trained at 408. A digital composite covariate (DCC) may be generated (e.g., based on the features, machine learning framework, and/or training) at 410 and may be based on baseline-clinical trial data (e.g., predictors and/or anticipated endpoints) from 412. A predicted outcome may be generated at 414 based on the DCC of 410 and statistical inference modeling may be used to extract baseline covariates at 416. The baseline covariates may be used to improve treatment effect estimates (e.g., by reducing sample sizes for clinical trials, targeting improved participants/features, etc.) at 418.

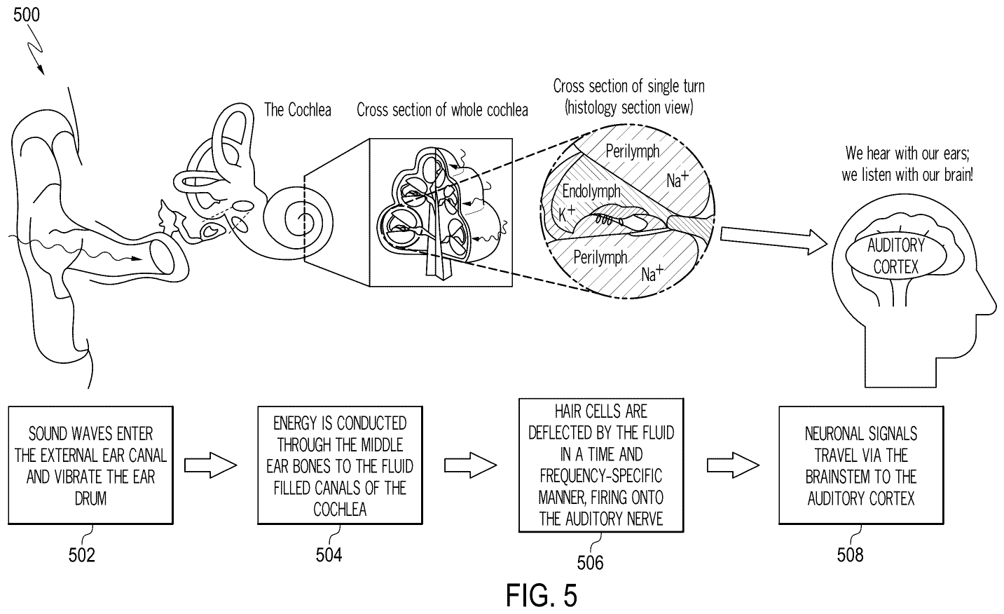

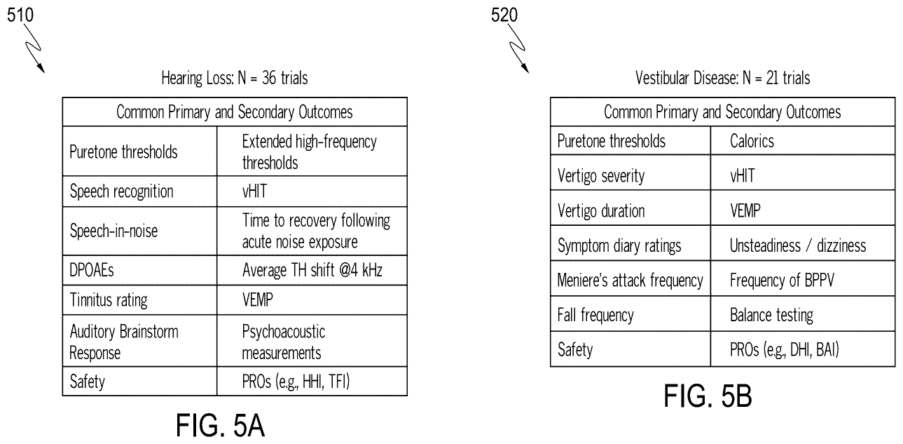

FIGS. 5A-12 are directed to an example auditory implementation of the techniques disclosed herein. According to this example implementation, auditory phenotyping may be formed using the techniques and systems disclosed herein. Such auditory phenotyping may be implemented using, for example, publically available datasets to improve clinical trials. Techniques disclosed herein may be used to perform clinical trials directed to, for example, test and/or validate therapeutics for medical conditions such as hearing loss. Such therapeutics may be favorable in comparison to conventional medical devices such as hearing aids and cochlear implants which may not restore hearing to a normal level and for which some individuals may not be candidates.

For example, digital endpoints may enhance phenotyping for auditory clinical trials based on frequent monitoring, diversified patient population, reduction in patient and/or clinical site burden, discerning a pharmacological effect, and a smaller population required for concept trials. Although auditory implementations are exemplified herein, it will be understood that techniques disclosed herein may be applied to any implementation such as, but not limited to, any human or animal therapeutic categories, medical categories, treatment categories, etc. For example, techniques disclosed herein may be applied to analysis, studies, treatments, etc. associated with any applicable disease, medical condition, physiological condition, movement condition, organ condition, psychological condition, sense-based condition, electrical data, chemical data, and/or the like, or a combination thereof.

A review of conventional clinical trials in hearing loss and vestibular disease is depicted in FIG. 5A and FIG. 5B. Of the 58 trails reviewed, only one used digital health technology as a secondary endpoint. This single trial used remote audiometry as a secondary outcome measure. One other trial in chronic inflammation used remote audiometry. FIG. 5A shows a table 510 summarizing 36 hearing loss trials and their respective common primary and secondary outcomes. FIG. 5B shows a table 520 summarizing 21 vestibular disease trials and their respective common primary and secondary outcomes. The trials summarized in table 510 and 520 did not consider digital health technology as a secondary endpoint.

Remote hearing assessments, such as those described further herein, have several advantages over conventional assessments. Such assessments include, but are not limited to, use of remote audiologic assessment tools (e.g., for online hearing screening, self-administered screening, mobile device-based self-administered screening, web-based remote diagnostic audiometric testing, clinician-administered screening), access to hard to reach populations (e.g., populations that may not be able to perform an in-person screening due to factors such as age, limited access to healthcare, remote location, mobility issues, fear of exposure to diseases, etc.), and being built to scale (e.g., via numerous language translations, via web-based tests deployed on servers local to a participant, etc.). Remote assessment validation studies show favorable agreement with conventional methods and favorable test-retest reliability. Experimental studies, such as those discussed herein for clinical trials, may be used to develop phenotyping tools and inform auditory clinical trials by providing overlap between technology validation studies, genome wide association studies, expansion phenotyping studies, and/or cohort studies.

Quality of life is a clinically meaningful outcome of auditory disorders that can be used to improve clinical trials. Improvement to quality of life may be exhibited by factors such as physiological factors (e.g., depression/anxiety, mood, etc.), perceived handicaps (e.g., hearing handicap, tinnitus handicap, etc.), physical function (e.g., gait characteristics, activity intensity, etc.), sleep quality (e.g., diurnal characteristics, tinnitus, etc.), social interaction (e.g., time alone (silence), time spent consuming content, cognitive function (e.g., working memory, speech-in-noise), and/or the like.

FIG. 5C shows a flow diagram 500 for how hearing is generally conducted for individuals. As depicted in flow diagram 500, sound waves enter an external car anal and vibrate an ear drum at 502. Energy is conducted through car bones to fluid filled canals of a cochlea at 504. Hair cells are deflected by the fluid in time and frequency-space, firing into an auditory nerve at 506. Neuronal signals travel via a brainstem to an auditory cortex at 508.

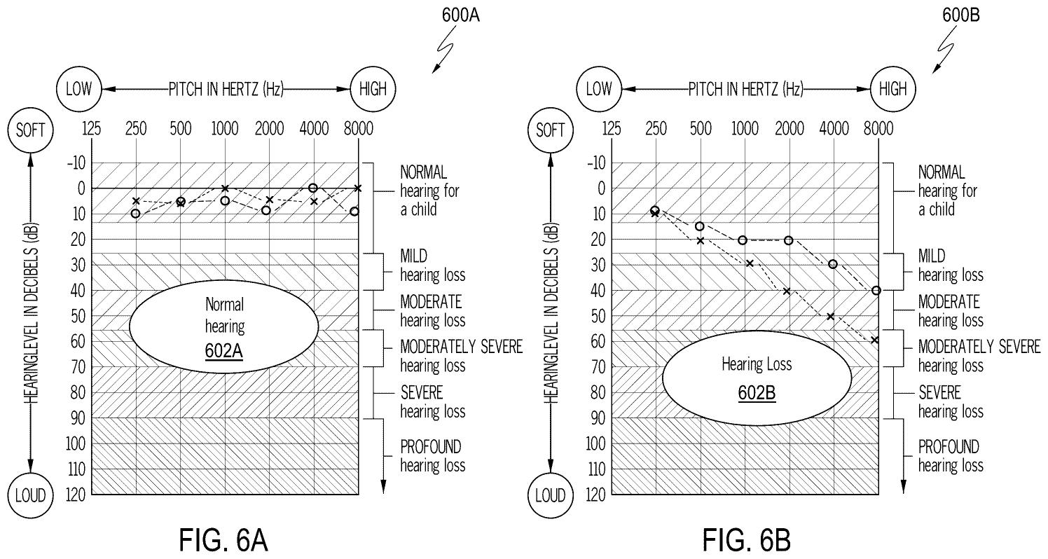

FIG. 6 shows chart 600A for a normal hearing assessment and chart 600B for a hearing quality assessment. The hearing assessment may be based on pure tone averages (PTAs) which may be the average of hearing levels at, for example, 500 Hz, 1000 Hz, 2000 Hz, and 4000 Hz. As shown in charts 600A and 600B, hearing may be assessed based on hearing levels registered by individuals for given pitches. The registered hearing levels may determine whether an individual experiences normal hearing 602A and/or hearing loss 602B. The assessments shown in FIG. 6 may be used to generate external data, as discussed herein.

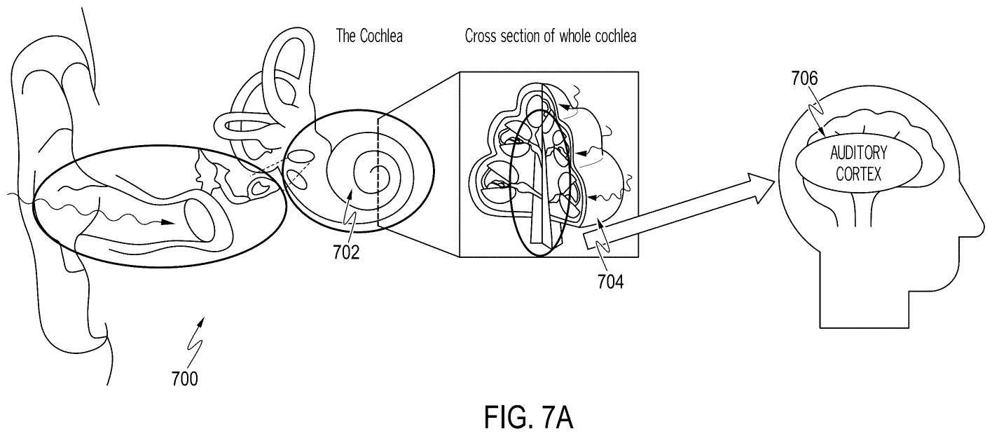

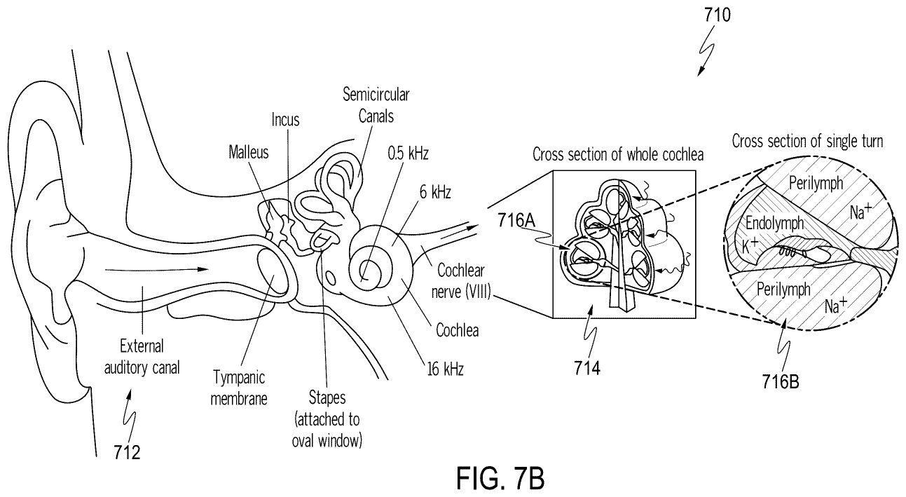

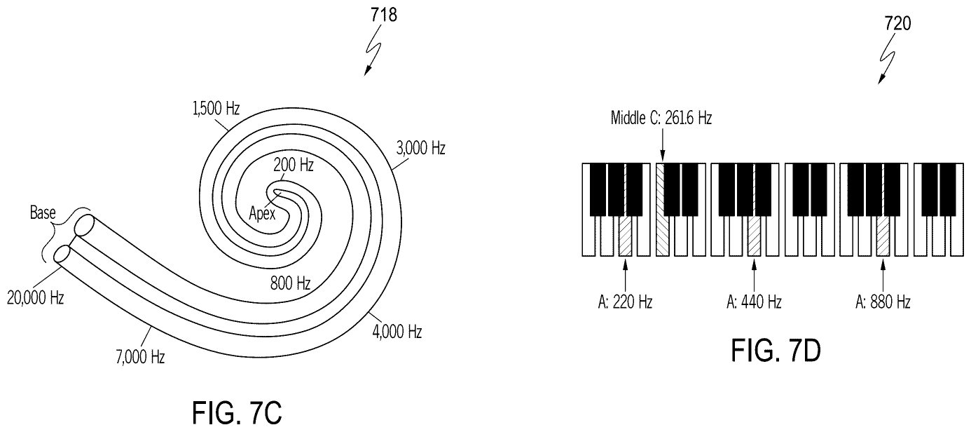

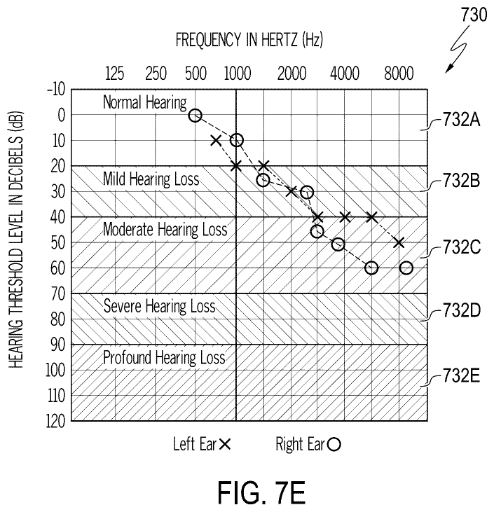

FIG. 7A shows a diagram 700 for characterizing hearing loss using multiple factors including mechanisms (e.g., conductive (outer and/or middle car), sensorineural (inner car), mixed, auditory neuropathy (central nervous system), age-of-onset/cause (e.g., congenital, pre-/post-lingual, early onset, age-related, noise-induced), onset and progression (e.g., sudden onset, progressive), frequencies (e.g., high frequency (most common), low frequency, cookie bite, noise notch, flat), and/or location (e.g., unilateral vs. bilateral). Hearing loss is characterized by one or more factors describing how, when, and/or where damage to the auditory system has occurred. Alternatively, or in addition, hearing loss may be characterized based on, for example, deficiencies in or more components of such a hearing path. The hearing loss characterizations of FIG. 7A may be used to generate external data, as discussed herein. Diagram 700 shows areas of a hearing path including the cochlea 702 including a cross section of a cochlea 704, and an auditory cortex 706.

FIG. 7B shows a diagram 710 that depicts anatomical components that contribute to hearing including a cochlea 712. A cross section 714 of the cochlea includes a single turn 716A which is shown via an expanded cross section of the single turn 716B. The cochlea 712 is a sensory organ that processes sound. The basilar membrane 718 shown in FIG. 7C is located inside the cochlea. The basilar membrane 718 is organized tonotopically by frequency, as depicted in FIG. 7C. For example, the basilar membrane 718 is organized tonotopically in a manner similar to the keys of a piano, as depicted at 720 of FIG. 7D. The anatomical components shown in FIGS. 7A-7D allow hearing to occur.

Audiogram 730 of FIG. 7E depicts an example implementation of how hearing is measured. As shown, audiogram 730 measures hearing threshold levels in decibels (dB) against frequency in hertz (Hz) Audiogram 730 is a picture of an individual's hearing sensitivity across the range of sound frequencies present in human speech. Audiogram 730 shows an example of how loud a particular frequency is required to be before the individual can hear a sound at the particular frequency. Audiograms such as audiogram 730 may be generated by an individual listening to sounds presented through headphones (e.g., in an acoustic sound booth) and indicates a response when a sound at a given frequency having a given decibel level is heard. Audiogram 730 depicts normal hearing at 732A, mild hearing loss at 732B, moderate hearing loss at 732C, severe hearing loss at 732D, and profound hearing loss at 732E, where each category corresponds to a hearing threshold level. As discussed herein a PTA may be the average of hearing levels at, for example, 500 Hz, 1000 Hz, 2000 Hz, and 4000 Hz. PTA approximates speech reception thresholds and may be used as a summary of the audiogram for each car.

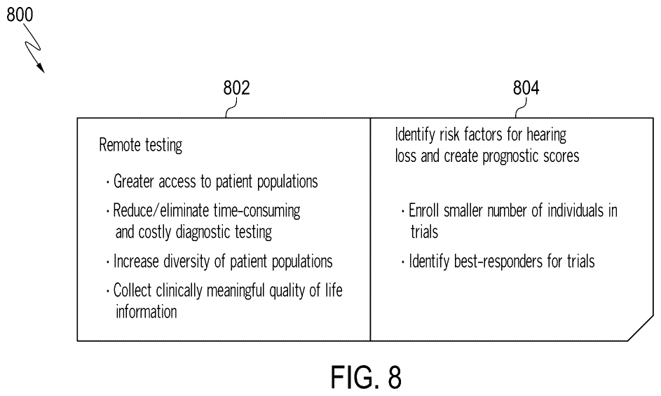

FIG. 8 shows a table 800 for enhancing auditory phenotyping for clinical trials. As shown in table 800, remote testing 802 may provide greater access to patient populations, reduce/eliminate resource intensive diagnostic testing, increase divert of clinical trial patient populations, and/or collect clinical meaningful quality of life information. The techniques disclosed herein may allow identification of baseline covariates that may indicate remote testing. For example, if identified baseline covariates include demographic information and survey responses, all or parts of a clinical trial may be implemented remotely as in person testing may not be required. Identification of such baseline covariates may reduce sample size requirements and may further increase access to potential clinical trial participants via remote testing. Accordingly, by identifying risk factors for hearing loss and applying a prognostic score implementation shown at 804, as discussed herein, a smaller number of individuals may be identified for a clinical trial and more applicable clinical trial participants may be identified.

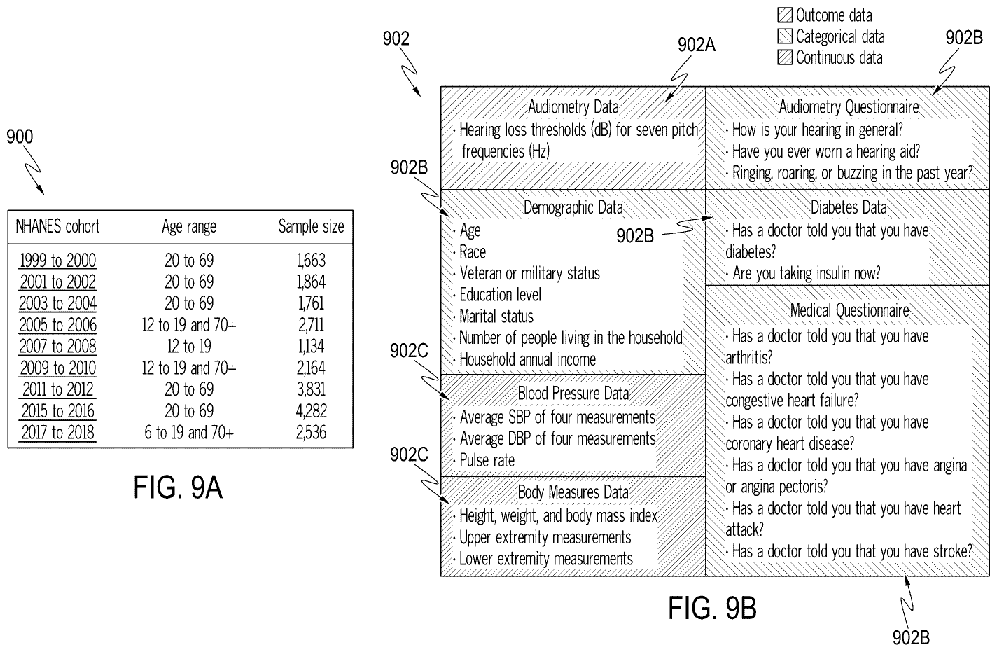

FIG. 9A shows table 900 for cohorts identified from a NHANES database. Such cohorts are identified based on a NHANES database cohort group, an age range, and a sample size. FIG. 9B shows table 902 of external data including outcome data 902A and categorical data 902B (feature data) and continuous data 902C (feature data) (e.g., where 902B and 902C correspond to external data received at step 202 of FIGS. 2A and 2B). Table 900 and 902 correspond to an experiment conducted in accordance with the techniques disclosed herein. As shown in table 902, the feature data includes categorical data 902B including demographic data, audiometry questionnaire data, diabetes data, medical questionnaire data, as well as continuous data 902C including blood pressure data and body measures data.

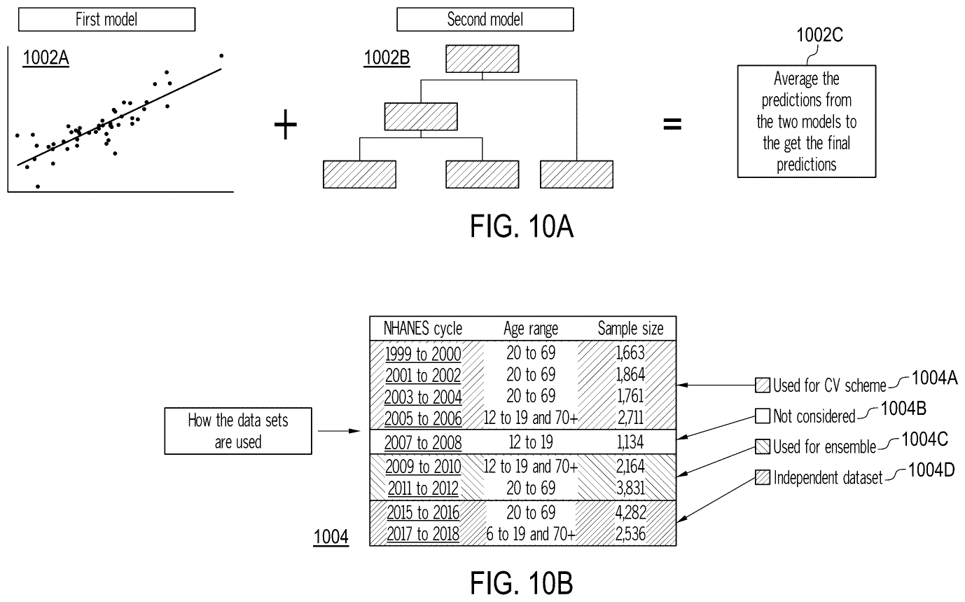

FIG. 10A shows an ensemble prediction framework (machine learning framework) that combines predictions from a first machine learning model 1002A and a second machine learning model 1002B. The first machine learning model 1002A and second machine learning model 1002B may be any applicable machine learning model such as those further disclosed herein. The combined data 1002C is combined in accordance with techniques disclosed herein. As shown, combined data 1002C may average the predictions form first machine learning model 1002A and second machine learning model 1002B. Table 1004 of FIG. 10B shows how the data of tables 900 and 902 is applied in accordance with the techniques disclosed herein. As shown, a first subset 1004A (first four rows) of the cohorts of table 900 are used to train the first machine learning model 1002A and second machine learning model 1002B (e.g., at step 204 of FIGS. 2A and 2B). The cohort 1004B corresponding to the fifth row is excluded. The cohort 1004C corresponding to the sixth and seventh rows are used to ensemble (e.g., stack) the first machine learning model 1002A and second machine learning model 1002B. A second subset of data 1004D (for the cohorts corresponding to the eighth and ninth rows) is used to validate machine learning framework in accordance with the techniques discussed herein (e.g., at step 206 of FIGS. 2A and 2B).

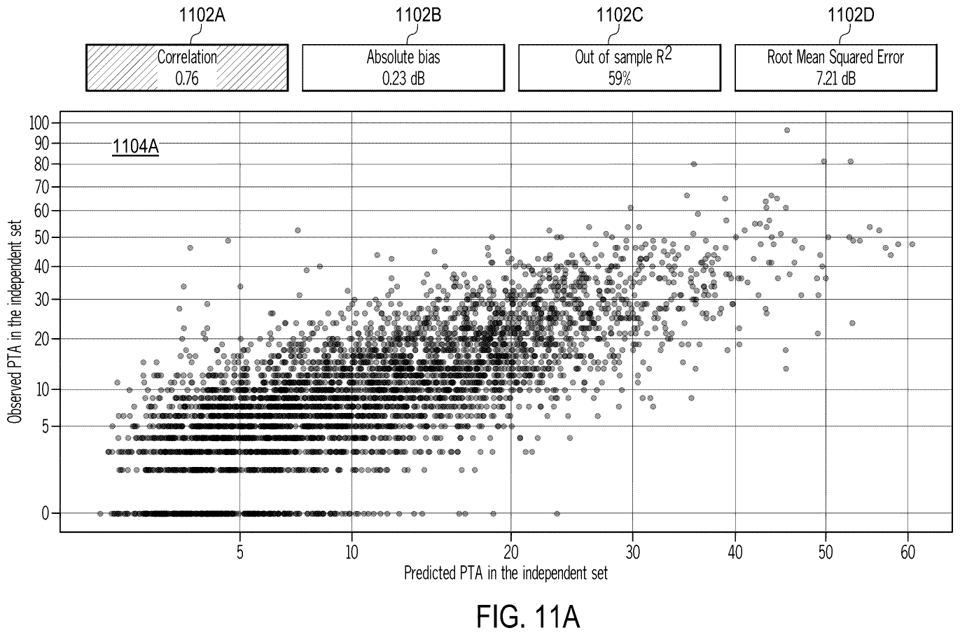

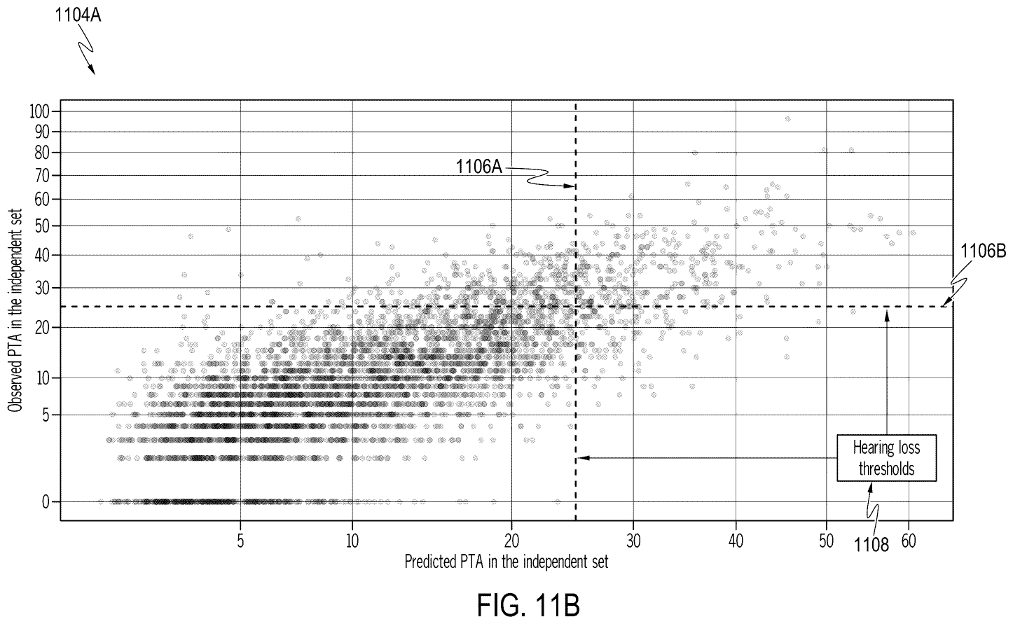

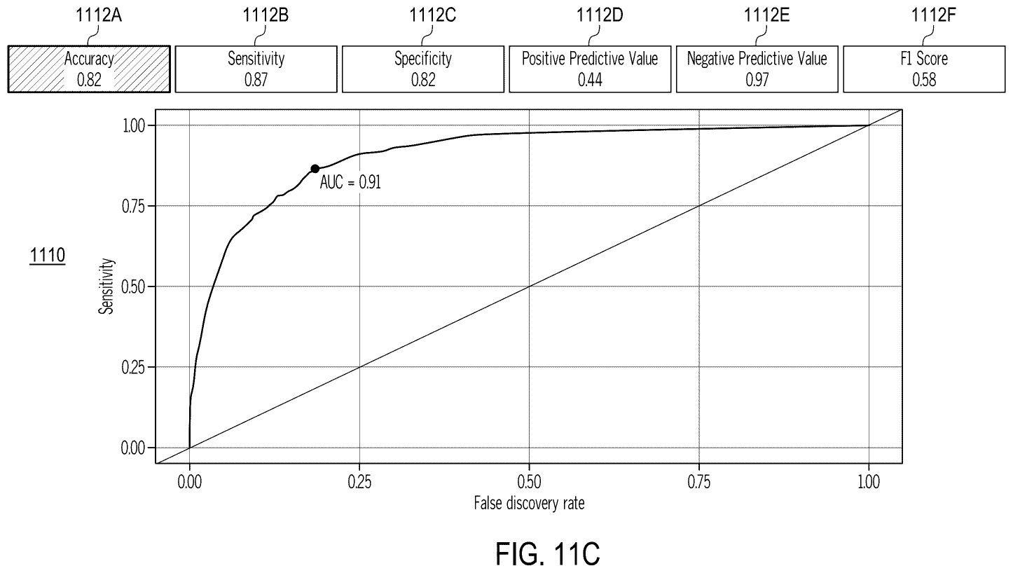

FIG. 11A shows a plot 1104 of performance metrics based on the validation of the machine learning framework depicted in FIG. 10A. As shown, the machine learning framework outputs results for the second subset of data having a correlation 1102A of 0.76 (e.g., as determined at step 220 of FIG. 2B), an absolute bias 1102B of 0.23 dB, an out of sample R² value 1102C of 59% and a root mean squared error 1102D of 7.2 dB. The machine learning framework of FIG. 10A is validate based on the performance metrics meeting one or more respective thresholds. As discussed herein, baseline covariates (e.g., as shown in FIG. 3) are determined based on the features most relied upon by the machine learning framework of FIG. 10A. FIG. 11B shows an annotated plot 1104A corresponding to plot 1104 of FIG. 11A. As shown in plot 1104A, hearing loss thresholds 1108 are designated based on an observed PTA in the independent set via threshold line 1106B and based on a predicted PTA in the independent set via threshold line 1106A. A PTA value of 25 dB for threshold line 1106B is used as the threshold for hearing loss. PTA may be categorized using this threshold to assess a model's ability to predict no hearing loss verses some hearing loss. According to an implementation, data points above threshold line 1106B and greater than threshold line 1106A or data points below threshold line 1106B and less than threshold line 1106A may be considered correctly classified whereas other data points may be considered incorrectly classified.

FIG. 11C shows a plot 1110 depicting the accuracy of prediction of a hearing loss class for individuals associated with the independent set shown in FIGS. 11A-11B. As shown, the model predicted the hearing loss class of 82% of the subjects in the independent set. The results shown in plot 1110 depict an accuracy 1112A of 0.82, a sensitivity 1112B of 0.87, a specificity 1112C of 0.82, a positive prediction value 1112D of 0.44, a negative prediction value 1112E of 0.97, and an F1 score 1112F of 0.58.

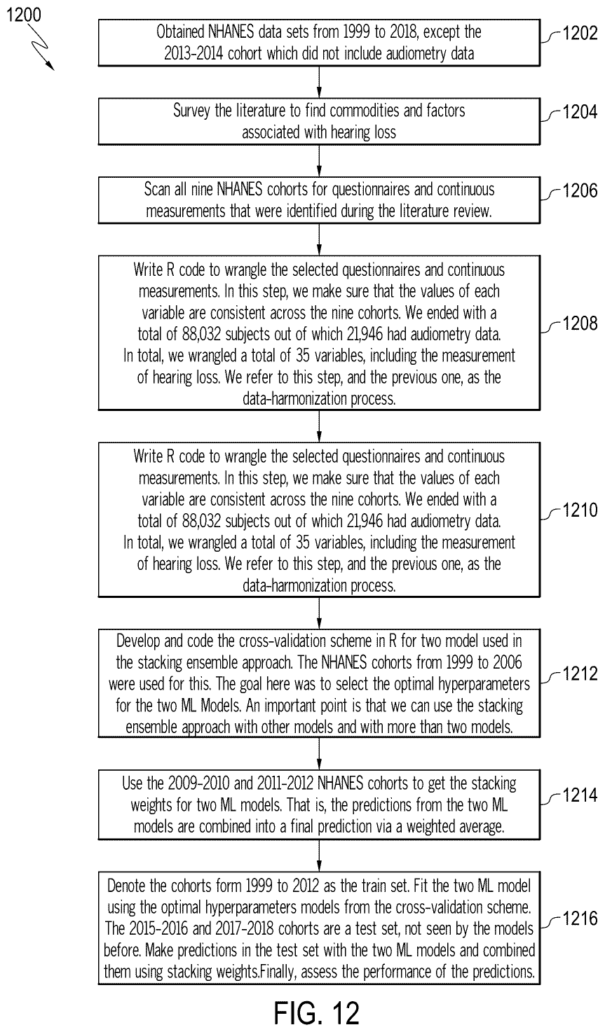

FIG. 12 shows flow diagram 1200 for an example experimental auditory implementation of the techniques disclosed herein at FIGS. 5-11C. As shown in flow diagram 1200, NHANES data (external data) including audiometry data (outcome data) for various cohorts may be obtained at 1202. Comorbidities and factors (features) associated with hearing loss may be identified at 1204. Cohorts for measurements (e.g., questionnaires and/or continues measurements) may be identified at 1206. The NHANES data may be scanned and wrangled (harmonized) to identify applicable external data (e.g., data associated with individuals having measured hearing quality data) at 1206, 1208, and 1210. A machine learning framework using a stacking ensemble approach may be developed and validated to assess performance of predictions output by the machine learning framework at 1212, 1214, and 1216, in accordance with techniques disclosed herein.

According to embodiments of the disclosed subject matter, publically available data such as NHATS audiometry data may be used in accordance with techniques disclosed herein. Such NHATS data may be collated and may be harmonized with NHANES data sets, such as those discussed herein. NHATS data may be used as another independent set to test the machine learning frameworks discussed herein. Further, use of digital composite covariates may be evaluated for one or more other medical conditions (e.g., with publically available data sets).

Each block in figures included herein including diagrams, flowcharts, flow diagrams, systems, etc. can represent a module, segment, or portion of program instructions, which includes one or more computer executable instructions for implementing the illustrated functions and operations. In some alternative implementations, the functions and/or operations illustrated in a particular block of a flow diagram or flowchart can occur out of the order shown in the respective figure.

For example, two blocks shown in succession can be executed substantially concurrently, or the blocks can sometimes be executed in the reverse order, depending upon the functionality involved. It will also be noted that each block of the flow diagram and combinations of blocks in the block can be implemented by special purpose hardware-based systems that perform the specified functions or acts, or combinations of special purpose hardware and computer instructions.

In various implementations disclosed herein, systems and methods are described for using machine learning to, for example, predict outcomes, determine prognostic scores, etc. By training a machine learning model, e.g., via supervised or semi-supervised learning, to learn associations between training data and ground truth data, the trained machine learning model may be used to validate outcomes, determine correlations, determine prognostic scores, etc.

A machine learning model may be implemented in accordance with techniques understood by one skilled in the art. As non-limiting examples, a machine learning model may encompass, but is not limited to, instructions, data, and/or a model configured to receive an input, and may apply one or more of a weight, bias, classification, or analysis on the input to generate an output. The output may include, for example, a classification of the input, an analysis based on the input, a design, process, prediction, or recommendation associated with the input, or any other suitable type of output. A machine learning model may be generally trained using training data, e.g., experiential data and/or samples of input data, which are fed into the model in order to establish, tune, or modify one or more aspects of the model, e.g., the weights, biases, criteria for forming classifications or clusters, or the like. Aspects of a machine learning model may operate on an input linearly, in parallel, via a network (e.g., a neural network), or via any suitable configuration.

The execution of the machine learning model may include deployment of one or more machine learning techniques, such as linear regression, logistical regression, least absolute shrinkage and selection operator (LASSO), extreme gradient boosting (XGBoost), tree-based model, random forest, gradient boosted machine (GBM), deep learning, and/or a deep neural network. Supervised and/or unsupervised training may be employed. For example, supervised learning may include providing training data and labels corresponding to the training data, e.g., as ground truth. Unsupervised approaches may include clustering, classification or the like. K-means clustering or K-Nearest Neighbors may also be used, which may be supervised or unsupervised. Combinations of K-Nearest Neighbors and an unsupervised cluster technique may also be used. Any suitable type of training may be used, e.g., stochastic, gradient boosted, random seeded, recursive, epoch or batch-based, etc.

As discussed herein, machine learning techniques may include one or more aspects according to this disclosure, e.g., a particular selection of training data, a particular training process for the machine learning model, operation of a particular device suitable for use with the trained machine learning model, operation of the machine learning model in conjunction with particular data, modification of such particular data by the machine learning model, etc., and/or other aspects that may be apparent to one of ordinary skill in the art based on this disclosure.

Generally, a machine learning model includes a set of variables, e.g., nodes, neurons, filters, etc., that are tuned, e.g., weighted or biased, to different values via the application of training data. In supervised learning, e.g., where a ground truth is known for the training data provided, training may proceed by feeding a sample of training data into a model with variables set at initialized values, e.g., at random, based on Gaussian noise, a pre-trained model, or the like. The output may be compared with the ground truth to determine an error, which may then be back-propagated through the model to adjust the values of the variable.

Training may be conducted in any suitable manner, e.g., in batches, and may include any suitable training methodology, e.g., stochastic or non-stochastic gradient descent, gradient boosting, random forest, etc. In some aspects, a portion of the training data may be withheld during training and/or used to validate the trained machine learning model, e.g., compare the output of the trained model with the ground truth for that portion of the training data to evaluate an accuracy of the trained model. The training of the machine learning model may be configured to cause the machine learning model to learn associations between training data and ground truth data, such that the trained machine learning model is configured to determine an output in response to the input data based on the learned associations.

In various implementations, the variables of a machine learning model may be interrelated in any suitable arrangement in order to generate the output. For example, in some aspects, the machine learning model may include image-processing architecture that is configured to identify, isolate, and/or extract features, geometry, and or structure in one or more of the medical imaging data and/or the non-optical in vivo image data. For example, the machine learning model may include one or more convolutional neural network (“CNN”) configured to identify features in data, and may include further architecture, e.g., a connected layer, neural network, etc., configured to determine a relationship between the identified features in order to determine a location in the data.

In some instances, different samples of training data and/or input data may not be independent. Thus, in some aspects, the machine learning model may be configured to account for and/or determine relationships between multiple samples.

For example, in some aspects, the machine learning models described herein may include a Recurrent Neural Network (“RNN”). Generally, RNNs are a class of feed-forward neural networks that may be well adapted to processing a sequence of inputs. In some aspects, the machine learning model may include a Long Shor Term Memory (“LSTM”) model and/or Sequence to Sequence (“Seq2Seq”) model. An LSTM model may be configured to generate an output from a sample that takes at least some previous samples and/or outputs into account. A Seq2Seq model may be configured to, for example, receive a sequence of non-optical in vivo images as input, and generate a sequence of locations, e.g., a path, in the medical imaging data as output.

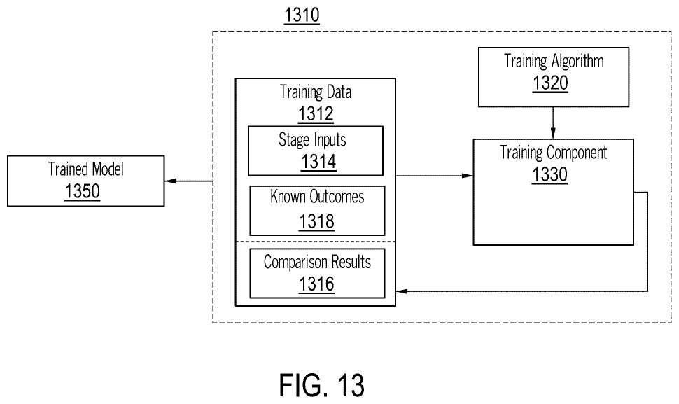

As disclosed herein, one or more implementations disclosed herein may be applied by using a machine learning model. A machine learning model as disclosed herein may be trained using one or more components or steps of FIGS. 1A-12. As shown in flow diagram 1310 of FIG. 13, training data 1312 may include one or more of stage inputs 1314 and known outcomes 1318 related to a machine learning model to be trained. The stage inputs 1314 may be from any applicable source including a component or set shown in the figures provided herein. The known outcomes 1318 may be included for machine learning models generated based on supervised or semi-supervised training. An unsupervised machine learning model might not be trained using known outcomes 1318. Known outcomes 1318 may include known or desired outputs for future inputs similar to or in the same category as stage inputs 1314 that do not have corresponding known outputs.

The training data 1312 and a training algorithm 1320 may be provided to a training component 1330 that may apply the training data 1312 to the training algorithm 1320 to generate a trained machine learning model 1350. According to an implementation, the training component 1330 may be provided comparison results 1316 that compare a previous output of the corresponding machine learning model to apply the previous result to re-train the machine learning model. The comparison results 1316 may be used by the training component 1330 to update the corresponding machine learning model. The training algorithm 1320 may utilize machine learning networks and/or models including, but not limited to a deep learning network such as Deep Neural Networks (DNN), Convolutional Neural Networks (CNN), Fully Convolutional Networks (FCN) and Recurrent Neural Networks (RCN), probabilistic models such as Bayesian Networks and Graphical Models, and/or discriminative models such as Decision Forests and maximum margin methods, or the like. The output of the flow diagram 1310 may be a trained machine learning model 1350.

A machine learning model disclosed herein may be trained by adjusting one or more weights, layers, and/or biases during a training phase. During the training phase, historical or simulated data may be provided as inputs to the model. The model may adjust one or more of its weights, layers, and/or biases based on such historical or simulated information. The adjusted weights, layers, and/or biases may be configured in a production version of the machine learning model (e.g., a trained model) based on the training. Once trained, the machine learning model may output machine learning model outputs in accordance with the subject matter disclosed herein. According to an implementation, one or more machine learning models disclosed herein may continuously update outputs based on feedback associated with use or implementation of the machine learning model outputs.

It should be understood that aspects provided in this disclosure are exemplary only, and that other aspects may include various combinations of features from other aspects, as well as additional or fewer features.

In general, any process or operation discussed in this disclosure that is understood to be computer-implementable, such as the processes illustrated in the flowcharts disclosed herein, may be performed by one or more processors of a computer system, such as any of the systems or devices in the exemplary environments disclosed herein, as described above. A process or process step performed by one or more processors may also be referred to as an operation. The one or more processors may be configured to perform such processes by having access to instructions (e.g., software or computer-readable code) that, when executed by the one or more processors, cause the one or more processors to perform the processes. The instructions may be stored in a memory of the computer system. A processor may be a central processing unit (CPU), a graphics processing unit (GPU), or any suitable types of processing unit.

A computer system, such as a system or device implementing a process or operation in the examples above, may include one or more computing devices, such as one or more of the systems or devices disclosed herein. One or more processors of a computer system may be included in a single computing device or distributed among a plurality of computing devices. A memory of the computer system may include the respective memory of each computing device of the plurality of computing devices.

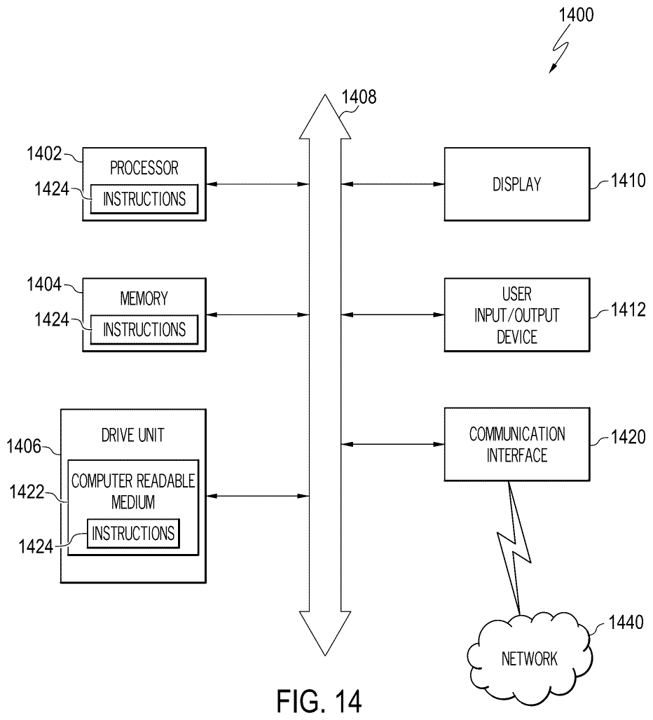

FIG. 14 is a simplified functional block diagram of a computer 1400 that may be configured as a device for executing the methods disclosed here, according to exemplary aspects of the present disclosure. For example, the computer 1400 may be configured as a system according to exemplary aspects of this disclosure. In various aspects, any of the systems herein may be a computer 1400 including, for example, a data communication interface 1420 for packet data communication. The computer 1400 also may include a central processing unit (“CPU”) 1402, in the form of one or more processors, for executing program instructions. The computer 1400 may include an internal communication bus 1408, and a storage unit 1406 (such as ROM, HDD, SDD, etc.) that may store data on a computer readable medium 1422, although the computer 1400 may receive programming and data via network communications (e.g., via network 1440). The computer 1400 may also have a memory 1404 (such as RAM) storing instructions 1424 for executing techniques presented herein, although the instructions 1424 may be stored temporarily or permanently within other modules of computer 1400 (e.g., processor 1402 and/or computer readable medium 1422). The computer 1400 also may include input and output ports 1412 and/or a display 1410 to connect with input and output devices such as keyboards, mice, touchscreens, monitors, displays, etc. The various system functions may be implemented in a distributed fashion on a number of similar platforms, to distribute the processing load. Alternatively, the systems may be implemented by appropriate programming of one computer hardware platform.

Program aspects of the technology may be thought of as “products” or “articles of manufacture” typically in the form of executable code and/or associated data that is carried on or embodied in a type of machine-readable medium. “Storage” type media include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer of the mobile communication network into the computer platform of a server and/or from a server to the mobile device. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to non-transitory, tangible “storage” media, terms such as computer or machine “readable medium” refer to any medium that participates in providing instructions to a processor for execution.

While the presently disclosed methods, devices, and systems are described with exemplary reference to transmitting data, it should be appreciated that the presently disclosed aspects may be applicable to any environment, such as a desktop or laptop computer, a mobile device, a wearable device, an application, or the like. Also, the presently disclosed aspects may be applicable to any type of Internet protocol.

It will be apparent to those skilled in the art that various modifications and variations can be made in the disclosed devices and methods without departing from the scope of the disclosure. Other aspects of the disclosure will be apparent to those skilled in the art from consideration of the specification and practice of the features disclosed herein. It is intended that the specification and examples be considered as exemplary only.

Aspects of the present disclosure may include the following:

Item 1: A method comprising: receiving external data including respective observed outcome data for a first set of subjects; training a machine learning framework based on a first subset of the external data to generate a trained machine learning framework; validating the trained machine learning framework using a second subset of the external data; extracting a baseline covariate based on validating the trained machine learning framework; determining a prognostic score for a first subject of a second set of subjects based on the baseline covariate; and classifying the first subject as a clinical trial subject based on the prognostic score.

Item 2: The method of item 1, wherein the external data is received from one of a publicly available source, a previous clinical trial source, or a previously generated data source.

Item 3: The method of item 1, wherein the external data further includes feature data of a plurality of features.

Item 4: The method of item 1, further comprising harmonizing the external data.

Item 5: The method of item 1, wherein the machine learning framework comprises one or more machine learning models.

Item 6: The method of item 1, wherein the machine learning framework is an ensemble framework.

Item 7: The method of item 1, wherein validating the trained machine learning framework includes determining a correlation between a predicted second subset outcome and an observed second subset outcome.

Item 8: The method of item 7, wherein validating the trained machine learning framework further includes comparing the correlation to a correlation threshold.

Item 9: The method of item 1, wherein extracting the baseline covariate includes determining a most relied upon feature of a plurality of features.

Item 10: The method of item 1, wherein extracting the baseline covariate includes determining a feature of a plurality of external data features that meets a weight threshold.

Item 11: The method of item 1, wherein determining the prognostic score for the first subject includes providing a participant feature to one of a prognostic algorithm or a prognostic machine learning model.

Item 12: A system comprising: a data storage device storing processor-readable instructions; and a processor operatively connected to the data storage device and configured to execute the instructions to perform operations that include: receiving external data including respective observed outcome data for a first set of subjects; training a machine learning framework based on a first subset of the external data to generate a trained machine learning framework; validating the trained machine learning framework using a second subset of the external data; extracting a baseline covariate based on validating the trained machine learning framework; determining a prognostic score for a first subject of a second set of subjects based on the baseline covariate; and classifying the first subject as a clinical trial subject based on the prognostic score.

Item 13: The system of item 12, wherein the external data is received from one of a publicly available source, a previous clinical trial source, or a previously generated data source.

Item 14: The system of item 12, wherein the external data further includes feature data of a plurality of features.

Item 15: The system of item 12, wherein the machine learning framework comprises one or more machine learning models.

Item 16: The system of item 12, wherein extracting the baseline covariate includes determining a most relied upon feature of a plurality of features.

Item 17: The system of item 12, wherein extracting the baseline covariate includes determining a feature of a plurality of external data features that meets a weight threshold.

Item 18: A method comprising: receiving external data including respective observed outcome data for a first set of subjects; training a machine learning framework based on a first subset of the external data to generate a trained machine learning framework; validating the trained machine learning framework using a second subset of the external data; determining a correlation between a second observed outcome data of the second subset of the external data to a predicted outcome data output by the trained machine learning framework based on the second subset of the external data; and determining a reduced sample size for a study based on the correlation.

Item 19: The method of item 18, wherein the correlation is based on a relationship between the second observed outcome data and the predicted outcome data.

Item 20: The method of item 18, wherein the reduced sample size is based on an original sample size of the study.

What is claimed is:

1. A method comprising:
receiving external data including respective observed outcome data for a first set of subjects;
training a machine learning framework based on a first subset of the external data to generate a trained machine learning framework;
validating the trained machine learning framework using a second subset of the external data;
extracting a baseline covariate based on validating the trained machine learning framework;
determining a prognostic score for a first subject of a second set of subjects based on the baseline covariate; and
classifying the first subject as a clinical trial subject based on the prognostic score.

2. The method of claim 1, wherein the external data is received from one of a publicly available source, a previous clinical trial source, or a previously generated data source.

3. The method of claim 1, wherein the external data further includes feature data of a plurality of features.

4. The method of claim 1, further comprising harmonizing the external data.

5. The method of claim 1, wherein the machine learning framework comprises one or more machine learning models.

6. The method of claim 1, wherein the machine learning framework is an ensemble framework.

7. The method of claim 1, wherein validating the trained machine learning framework includes determining a correlation between a predicted second subset outcome and an observed second subset outcome.

8. The method of claim 7, wherein validating the trained machine learning framework further includes comparing the correlation to a correlation threshold.

9. The method of claim 1, wherein extracting the baseline covariate includes determining a most relied upon feature of a plurality of features.

10. The method of claim 1, wherein extracting the baseline covariate includes determining a feature of a plurality of external data features that meets a weight threshold.

11. The method of claim 1, wherein determining the prognostic score for the first subject includes providing a participant feature to one of a prognostic algorithm or a prognostic machine learning model.

12. A system comprising:
a data storage device storing processor-readable instructions; and
a processor operatively connected to the data storage device and configured to execute the instructions to perform operations that include:
receiving external data including respective observed outcome data for a first set of subjects;
training a machine learning framework based on a first subset of the external data to generate a trained machine learning framework;
validating the trained machine learning framework using a second subset of the external data;
extracting a baseline covariate based on validating the trained machine learning framework;
determining a prognostic score for a first subject of a second set of subjects based on the baseline covariate; and
classifying the first subject as a clinical trial subject based on the prognostic score.

13. The system of claim 12, wherein the external data is received from one of a publicly available source, a previous clinical trial source, or a previously generated data source.

14. The system of claim 12, wherein the external data further includes feature data of a plurality of features.

15. The system of claim 12, wherein the machine learning framework comprises one or more machine learning models.

16. The system of claim 12, wherein extracting the baseline covariate includes determining a most relied upon feature of a plurality of features.

17. The system of claim 12, wherein extracting the baseline covariate includes determining a feature of a plurality of external data features that meets a weight threshold.

18. A method comprising:
receiving external data including respective observed outcome data for a first set of subjects;
training a machine learning framework based on a first subset of the external data to generate a trained machine learning framework;
validating the trained machine learning framework using a second subset of the external data;
determining a correlation between a second observed outcome data of the second subset of the external data to a predicted outcome data output by the trained machine learning framework based on the second subset of the external data; and
determining a reduced sample size for a study based on the correlation.

19. The method of claim 18, wherein the correlation is based on a relationship between the second observed outcome data and the predicted outcome data.

20. The method of claim 18, wherein the reduced sample size is based on an original sample size of the study.
